(19) United States
(12) Patent Application Publication
(10) Pub. No.: US 2022/0328165 A1
(43) Pub. Date: Oct. 13, 2022

(54) INFORMATION PROCESSING APPARATUS, INFORMATION PROCESSING METHOD, AND PROGRAM

(71) Applicant: SONY GROUP CORPORATION, TOKYO (JP)

(72) Inventors: MASAYA TAKEMOTO, TOKYO (JP); YUKI SUGIE, TOKYO (JP)

(21) Appl. No.: 17/634,267

(22) PCT Filed: Aug. 5, 2020
(86) PCT No.: PCT/JP2020/030056

§ 371 Date: Feb. 10, 2022

(30) Foreign Application Priority Data
Aug. 20, 2019 (JP) 2019-150328

(51) Int. Cl. G16H 30/20; G06T 7/00; G16H 50/20

(52) U.S. Cl. CPC G16H 30/20; G06T 7/0012; G16H 50/20

(57) ABSTRACT

An information processing system includes a communication unit and a controller. The communication unit transmits a request to load a second pathological image different from a first pathological image being displayed. The controller causes a display unit to display related information related to a pathological diagnosis or an operation of a pathology medical device before the loaded second pathological image is displayed.

### TECHNICAL FIELD

The present technology relates to an information processing system capable of executing information processing in which digital pathological images stored in servers can be loaded and displayed in a digital pathology imaging (DPI) system, and to an information processing method and a program in the information processing system.

### BACKGROUND ART

In the field of pathology and the like, a system has been proposed in which an image of a cell, tissue, organ, or the like of a living body, which is obtained by an optical microscope, is changed into a digital image, and a doctor, a pathologist, or the like examines the tissue or the like or diagnoses a patient on the basis of the digital image.

For example, Patent Literature 1 discloses a system in which an observation target area is divided and imaged by a digital microscope, a plurality of partial images obtained is connected to generate a single pathological image, and the pathological image and a pathological index are simultaneously displayed.

### CITATION LIST

### Patent Literature

Patent Literature 1: Japanese Patent Application Laid-open No. 2014-66788

### DISCLOSURE OF INVENTION

### Technical Problem

However, since a pathological image is required to have high image quality, the file capacity of one image is very large. For that reason, when a pathologist checks a pathological image, it takes time for a viewer computer to download a pathological image from a server or read the downloaded pathological image, and it is not possible to immediately display the pathological image. As a result, there is a problem that the pathology workflow is interrupted.

In view of the above circumstances, it is an object of the present technology to provide an information processing system, an information processing method, and a program that are capable of effectively utilizing a time for loading a digital pathological image for a pathology workflow.

### Solution to Problem

In order to solve the problem described above, an information processing system according to an embodiment of the present technology includes a communication unit and a controller. The communication unit transmits a request to load a second pathological image different from a first pathological image being displayed. The controller causes a display unit to display related information related to a pathological diagnosis or an operation of a pathology medical device before the loaded second pathological image is displayed.

With this configuration, the information processing system can display information regarding a pathological diagnosis or an operation of a pathology medical device during a time for loading a digital pathological image, thus effectively utilizing such a time for a pathology workflow. Here, the communication unit and the controller may be incorporated in one device or may be separately incorporated in different devices.

The controller may display the related information during loading of the second pathological image.

The controller may switch the related information to the second pathological image when loading of the second pathological image is completed.

As a result, the information processing system can allocate the entire load time of the second pathological image to the display of the related information and can display the second pathological image immediately after the completion of the loading, thus preventing the interruption of the pathology workflow.

The controller may switch the related information to the second pathological image for display when loading of the second pathological image is completed and when an operation for displaying the second pathological image is received from a user.

As a result, the information processing system can make a transition to the display of the second pathological image after the pathologist checks the related information with certainty by requesting an explicit operation to switch the image.

The controller may acquire load time information indicating a time necessary for loading the second pathological image and display the related information of a different type or amount in accordance with the load time information.

As a result, the information processing system can prevent the pathologist from failing to sufficiently check the related information before the second pathological image is displayed or from having spare time by displaying an appropriate information amount of the related information in accordance with the load time of the second pathological image.

The controller may display, as the related information, information regarding the second pathological image.

The controller may display, as the related information, information indicating a clinical diagnosis result corresponding to the second pathological image.

As a result, the information processing system allows the pathologist to know a corresponding clinical diagnosis result as preliminary knowledge before a pathological diagnosis, thus preventing the clinical diagnosis result and a pathological diagnosis result from being mismatched and from causing a misdiagnosis.

The controller may display, as the related information, information indicating a sampling site of a specimen serving as an imaging source of the second pathological image.

As a result, the information processing system allows the pathologist to know the sampling site of the specimen as preliminary knowledge before a pathological diagnosis, thus preventing a misdiagnosis and sample mix-up.

The controller may display, as the related information, information indicating a patient serving as an imaging source of the second pathological image.

As a result, the information processing system allows the pathologist to know the patient information as preliminary knowledge before a pathological diagnosis, thus preventing sample mix-up.

The controller may display, as the related information, information indicating a diagnosis result by artificial intelligence (AI) regarding the first pathological image.

As a result, the information processing system allows the pathologist to know a corresponding diagnosis result by the AI after the pathological diagnosis regarding the first pathological image, thus preventing a misdiagnosis after the fact.

The controller may display, as the related information, information indicating a diagnosis result by artificial intelligence (AI) regarding the second pathological image.

As a result, the information processing system allows the pathologist to know a corresponding diagnosis result by the AI as preliminary knowledge before the pathological diagnosis regarding the second pathological image, thus preventing a misdiagnosis.

The controller may acquire first sample information indicating a sample serving as an imaging source of the first pathological image and second sample information indicating a sample serving as an imaging source of the second pathological image, and may display the related information when the first sample information and the second sample information are different from each other.

The controller may acquire first patient information indicating a patient serving as an imaging source of the first pathological image and second patient information indicating a patient serving as an imaging source of the second pathological image, and may display the related information when the first patient information and the second patient information are different from each other.

As a result, the information processing system does not constantly display the related information every time the pathological image is switched, but displays the related information only when a precondition of a sample or patient serving as an imaging source are changed, so that it is possible to provide the minimum necessary information for the diagnosis while preventing the pathologist from feeling troublesome.

An information processing method according to another embodiment of the present technology includes: transmitting a request to load a second pathological image different from a first pathological image being displayed; and causing a display unit to display related information related to a pathological diagnosis or an operation of a pathology medical device before the loaded second pathological image is displayed.

A program according to another embodiment of the present technology causes an information processing apparatus to execute the steps of: transmitting a request to load a second pathological image different from a first pathological image being displayed; and causing a display unit to display related information related to a pathological diagnosis or an operation of a pathology medical device before the loaded second pathological image is displayed.

### Advantageous Effects of Invention

As described above, according to the present technology, it is possible to effectively utilizing a time for loading a digital pathological image for a pathology workflow. However, this effect is not a limitation of the present technology.

### BRIEF DESCRIPTION OF DRAWINGS

FIG. 1 is a diagram showing a network configuration of a DPI system according to an embodiment of the present technology.

FIG. 2 is a diagram showing a more detailed network configuration of the DPI system and a database configuration of each device.

FIG. 3 is a block diagram showing a hardware configuration of a computer serving as a viewer in the DPI system.

FIG. 4 is a flowchart showing the operation flow of the DPI system.

FIG. 5 is a flowchart showing the operation flow of the DPI system according to another embodiment of the present technology.

FIG. 6 is a flowchart showing the operation flow of the DPI system according to another embodiment of the present technology.

FIG. 7 is a flowchart showing the operation flow of the DPI system according to another embodiment of the present technology.

FIG. 8 is a flowchart showing the operation flow of the DPI system according to another embodiment of the present technology.

FIG. 9 is a diagram showing an example of a user setting table used in the DPI system according to another embodiment of the present technology.

### Mode(s) for Carrying Out the Invention

Hereinafter, an embodiment according to the present technology will be described with reference to the drawings.

[System Configuration]

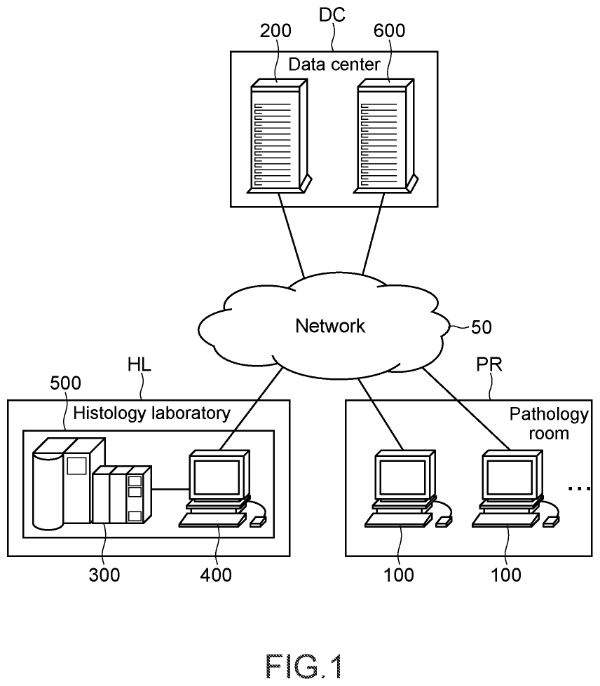

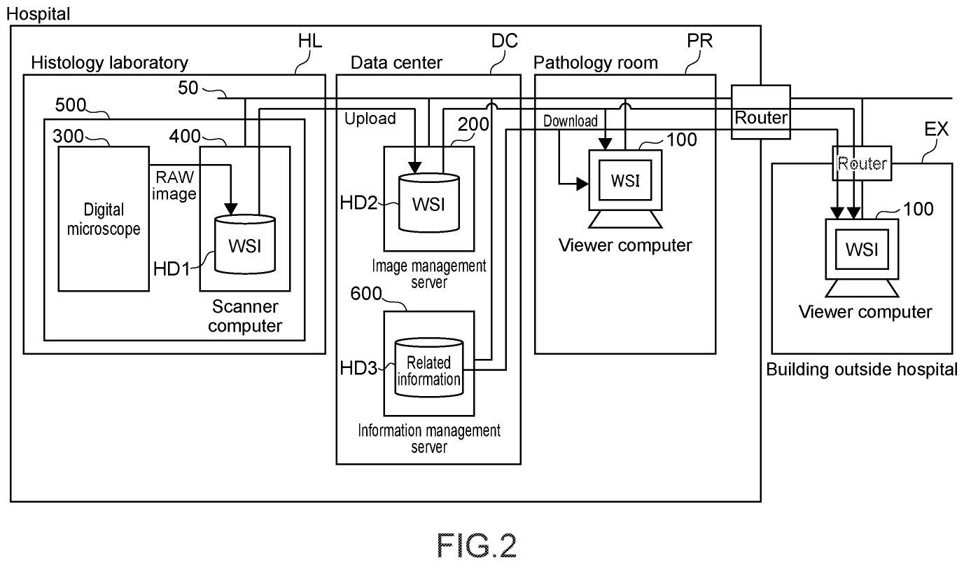

FIG. 1 is a diagram showing a network configuration of a DPI system according to this embodiment. Further, FIG. 2 is a diagram showing a more detailed network configuration of the DPI system and a database configuration of each device. Note that this system is not limited to the illustrated one, but may be configured by a system including one or more computers and one or more storage devices that store instructions to be implemented by one or more computers when executed by one or more computers.

As shown in both the figures, the DPI system is configured as a network in a hospital, for example. In the hospital, a histology laboratory HL, a data center DC, and a pathology room PR are present.

A scanner 500 is installed in the histology laboratory, an image management server 200 and an information management server 600 are installed in the data center, and a viewer computer 100 is installed in the pathology room. The viewer computer 100 may also be located in a building EX outside the hospital.

The scanner 500 includes a digital microscope 300 and a scanner computer 400. The digital microscope 300 images a pathological glass specimen (prepared slide) containing a stained section of a sample taken from a patient.

The scanner computer 400 performs image processing such as development processing, shading processing, color balance correction, gamma correction, and 8-bit processing on a RAW image of the imaged pathological glass specimen. Subsequently, the image is divided into tiles according to different resolutions, converted into, for example, a joint photographic experts group (JPEG) format, and compressed to be stored as a whole slide imaging (WSI) image in a hard disk HD1.

The WSI images stored in the hard disk HD1 of the scanner computer 400 are then uploaded over the network 50 to a hard disk HD2 of the image management server 200 at the data center DC in the same hospital. Each of the WSI images is associated with an image ID that identifies the WSI image.

A pathologist who is a WSI observer is in the pathology laboratory PR in the hospital or the building EX outside the hospital, downloads and observes a JPEG image stored in the hard disk HD2 of the image management server 200 by using the viewer computer 100 connected to the image management server 200 via the network 50, and performs a pathological diagnosis.

Further, a hard disk HD3 of the information management server 600 stores information related to a pathological diagnosis (hereinafter, referred to as related information). The related information includes, but is not limited to, electronic medical record information of patients, information indicating the sampling site of the specimen from which the WSI was created (sketch of the sampling site by a specimen creator), a pathological diagnosis application form, and the like.

The related information is linked to the WSI via an image ID, a patient ID, or the like. The related information is downloaded as appropriate when the pathologist performs a pathological diagnosis using the WSI image, for example, and is browsed using the viewer computer 100.

[Hardware Configuration of Viewer Computer]

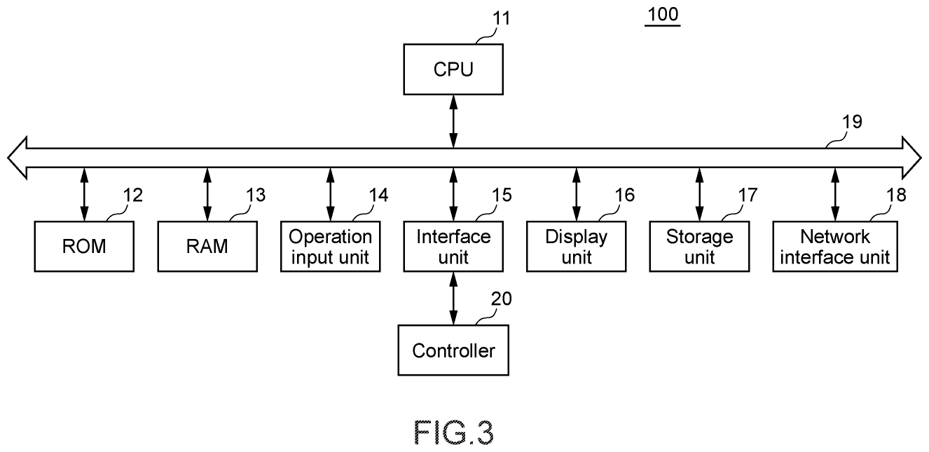

Next, a hardware configuration of the viewer computer 100 will be described. FIG. 3 is a block diagram showing a hardware configuration of the viewer computer 100.

As shown in the figure, the viewer computer 100 includes a central processing unit (CPU) 11 that performs arithmetic control, a read only memory (ROM) 12, a random access memory (RAM) 13 that serves as a work memory of the CPU 11, an operation input unit 14 that inputs an instruction corresponding to a user's operation, an interface unit 15, a display unit 16, a storage unit 17, a network interface unit 18, and a bus 19 that connects them to each other.

The ROM 12 stores programs for executing various types of processing. A controller 20 is connected to the interface unit 15. The controller 20 includes various buttons and sticks and is capable of receiving various inputs made by the user. The controller 20 also includes an acceleration sensor and an inclination sensor and is capable of receiving an instruction given to the controller 20 when the user tilts or shakes the controller 20.

The network interface unit 18 is, for example, a network interface card (NIC), and connects the viewer computer 100 to the network 50. A liquid crystal display, an 1 luminescence (EL) display, a plasma display, or the like is applied to the display unit 16. The display unit 16 may be provided integrally with the viewer computer 100, or may be connected to the outside of the viewer computer 100. A magnetic disk typified by a hard disk drive (HDD) or a semiconductor memory, an optical disc, or the like is applied to the storage unit 17. For example, various programs such as a program for the viewer of the WSI images are also stored in the storage unit 17.

The CPU 11 expands a program, which corresponds to an instruction given from the operation input unit 14 among a plurality of programs stored in the ROM 12, into the RAM 13, and controls the display unit 16 and the storage unit 17 as appropriate in accordance with the expanded program.

Although not shown in the figures, the hardware configurations of the image management server 200, the information management server 600, and the scanner computer 400 are also roughly the same as that of the viewer computer 100.

[Operation of System]

Next, the operations of the server and the viewer in the system configured as described above will be described. In this embodiment and other embodiments, the operations of the server and the viewer are performed in cooperation with the CPU and the above-mentioned software executed under the control of the CPU.

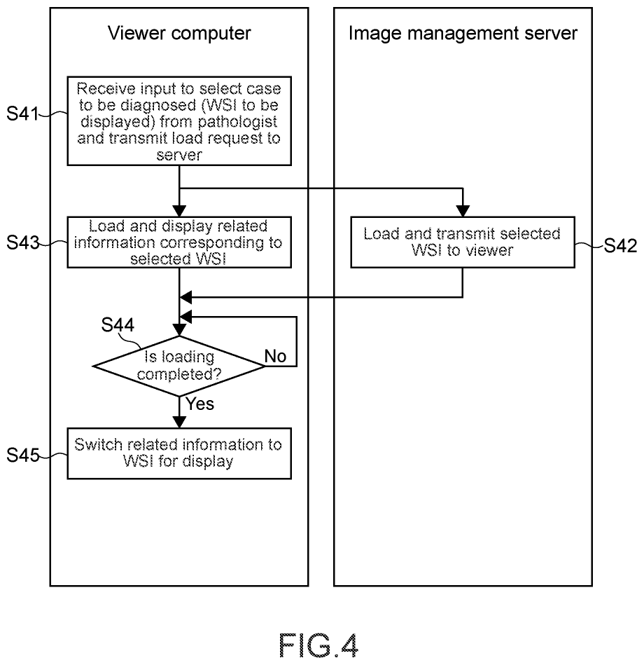

FIG. 4 is a flowchart showing a flow of WSI image display processing in the DPI system according to this embodiment.

As shown in the figure, first, the CPU 11 of the viewer computer 100 receives, after starting a program for the viewer, an input to select a case to be diagnosed (i.e., a WSI image to be displayed) from a pathologist as a user through the operation input unit 14, and transmits a load request for the selected WSI image to the image management server 200 (Step 41).

Upon receiving the load request, the image management server 200 loads the selected WSI image from the HD2 with reference to the image ID of the WSI image included in the load request and transmits the selected WSI image to the viewer computer 100 (Step 43).

Meanwhile, the CPU 11 of the viewer computer 100 downloads the related information corresponding to the selected WSI image from the HD3 of the information management server 600 and causes the display unit 16 to display the downloaded related information (Step 43).

Here, the related information to be displayed includes, as described above, information indicating clinical diagnosis contents corresponding to the selected WSI image, information indicating the sampling site of the specimen from which the selected WSI image was created, and information regarding a patient to be diagnosed using the selected WSI image (name, patient ID, etc.).

The clinical diagnosis contents are displayed as the related information, and thus a misdiagnosis that may occur due to inadequate excision of the specimen, insufficient knowledge or fatigue of a pathologist, or the like can be prevented. In other words, if the clinical diagnosis contents and a pathological diagnosis result based on the WSI image do not coincide with each other, it is generally recommended to carry out the pathological diagnosis again on the basis of various suspicious reasons. Therefore, the DPI system can prevent a misdiagnosis by inputting corresponding clinical diagnosis contents to a pathologist before the pathologist observes a WSI image.

Further, the information of the sampling site of the specimen (sketch of the sampling site by a specimen creator or the sampling site described in a pathological diagnosis application form) is displayed as the related information, and thus the pathologist can check the sampling site of the specimen before performing a pathological diagnosis, thus preventing a misdiagnosis or sample mix-up. Further, the patient information is displayed as the related information, and thus the sample is prevented from being mixed up.

Subsequently, the CPU 11 determines whether the download of the selected WSI has been completed (Step 44).

If it is determined that the download of the WSI image is completed, the CPU 11 switches the related information displayed on the display unit 16 to the WSI for display (Step 45).

Subsequently, the DPI system repeats the above processing each time an input to select a WSI is received from the pathologist. In other words, after displaying the selected WSI image (first pathological image), the viewer computer 100 transmits a load request to the image management server 200 each time a WSI image (second pathological image) of the same sample (patient) having a different display range and resolution or a WSI image (second pathological image) of another sample (patient) is selected by the pathologist, displays the related information until the loading is completed, and displays the WSI image (second pathological image) when the loading is completed.

Here, the trigger by which the load request is to be transmitted is not limited to the case where the pathologist explicitly selects a WSI image. For example, if the pathologist inputs an operation to close the WSI image being displayed and if a WSI image of the same sample exists, a load request for that WSI image may be automatically transmitted.

[Effects]

As described above, in this embodiment, the DPI system can display related information during a time for loading a digital pathological image, thus effectively utilizing such a time for the pathology workflow, and also reducing the stress of the pathologist on a long load time. Further, the pathologist can make a more correct diagnosis by referring to the related information.

[Modifications]

The present technology is not limited to the embodiment described above, and various modifications may be made without departing from the gist of the present technology.

(Modification 1 Regarding Method of Displaying Related Information: Switching of Related Information Based on Load Time)

In the embodiment described above, the CPU 11 of the viewer computer 100 uniformly displays the related information during loading of a WSI image. However, since the load time differs depending on the manufacturer or the like of the scanner 500, the pathologist may not be able to check a large amount of related information even if such a large amount of related information is displayed for the WSI images having a short load time. Further, the pathologist may have too much spare time even if a small amount of related information is displayed for the WSI images having a long load time.

Therefore, the DPI system may change the information to be displayed depending on the calculated or preset estimated load time. For example, the viewer computer 100 may display only the sketch of a sampling position of a specimen if the load time is assumed to be short, and may also display comments regarding the specimen in addition to the sketch if the load time is assumed to be long.

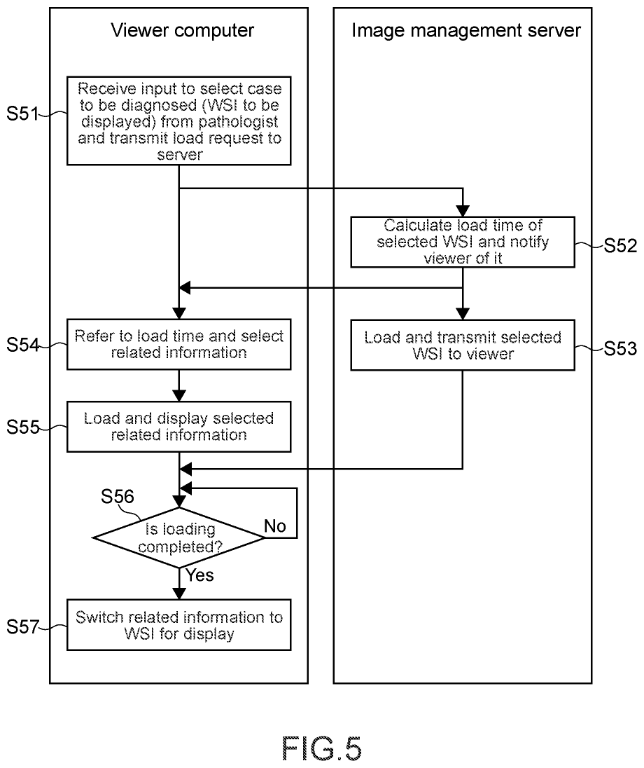

FIG. 5 is a flowchart showing the flow of processing of displaying a WSI image of the DPI system when the load time is calculated.

For example, the image management server 200 calculates an estimated load time on the basis of the file size of the selected WSI image and the communication speed (bandwidth) of the network interface unit 18, and notifies the viewer computer of the calculated load time (Step 52). The CPU 11 of the viewer computer 100 refers to the calculated load time, selects the related information of a corresponding type or amount (Step 54), and loads and displays it (Step 55). Other processes are the same as those shown in FIG. 4.

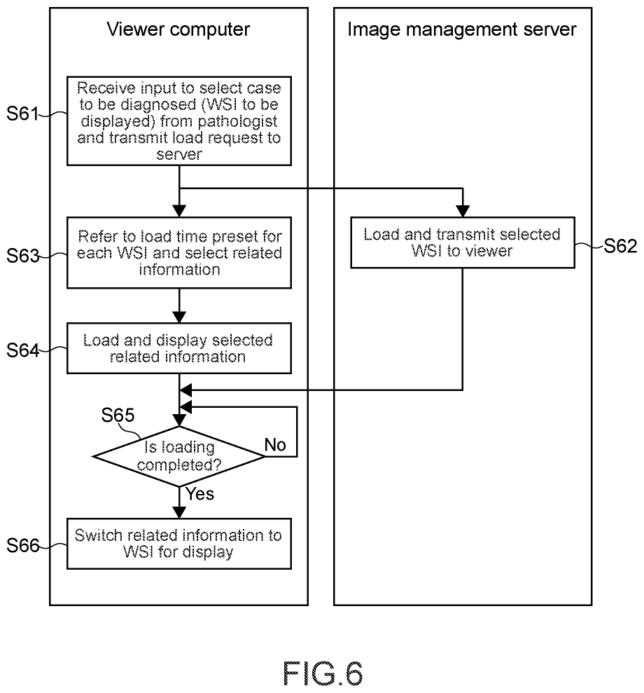

FIG. 6 is a flowchart showing the flow of processing of displaying a WSI image of the DPI system when the load time is set in advance.

The CPU 11 of the viewer computer 100 refers to the preset load time for the selected WSI image, for example, on the basis of the file size, a rough communication speed, or the like, selects the related information of a corresponding type or amount (Step 63), and loads and displays it (Step 64). Other processes are the same as those shown in FIG. 4.

(Modification 2 Regarding Method of Displaying Related Information: Switching of Related Information on Basis of User Setting)

The causes of the misdiagnosis or sample mix-up tend to differ from hospital to hospital. When a close call or incident occurs as an activity of each hospital, it is necessary to take countermeasures to prevent the same from happening in the future. As one of the countermeasures, the contents of the related information to be displayed as preventive countermeasures against the close call that has occurred in the past can be switched on the basis of the user setting.

For example, in hospitals in which many misdiagnoses occur, clinical diagnosis contents may be displayed as the related information, and in hospitals in which sample mix-up occurs many times, patient information and specimen-sampling-site information may be displayed as the related information. The information regarding these user settings is stored in, for example, the HD3 of the information management server or the storage unit 17 of the viewer computer 100.

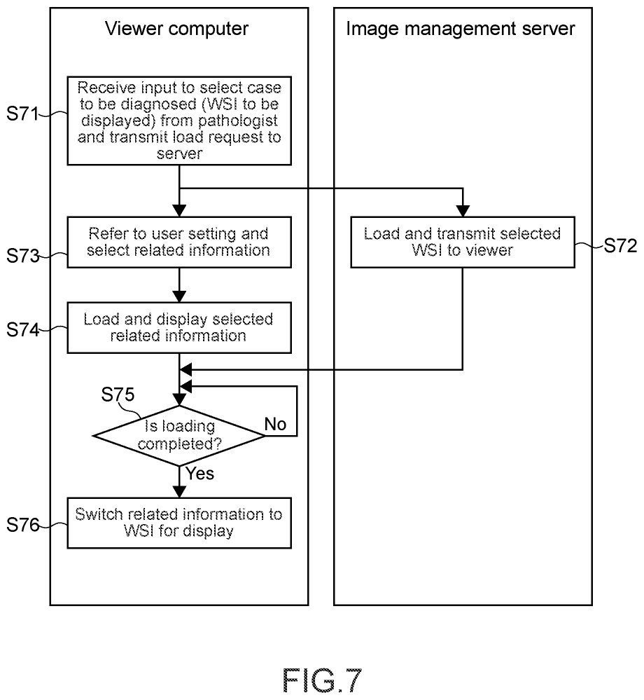

FIG. 7 is a flowchart showing a flow of processing of displaying a WSI image of the DPI system in this case.

The CPU 11 of the viewer computer 100 refers to the user setting information, selects related information of the currently set type (Step 73), and loads and displays it (Step 74). Other processes are the same as those shown in FIG. 4.

(Modification 3 Regarding Method of Displaying Related Information: Combination of Modifications 1 and 2)

Modifications 1 and 2 may be combined. In other words, the DPI system categorizes the display contents of the related information, the user selects the category to be displayed, and the viewer computer 100 determines the display contents of the related information by referring to a load time and a user setting table during loading of a WSI image from the image management server 200.

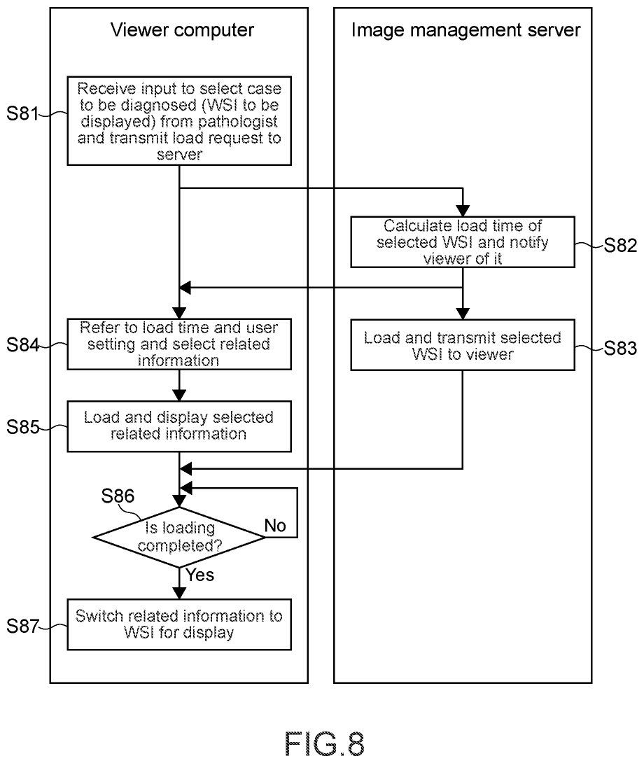

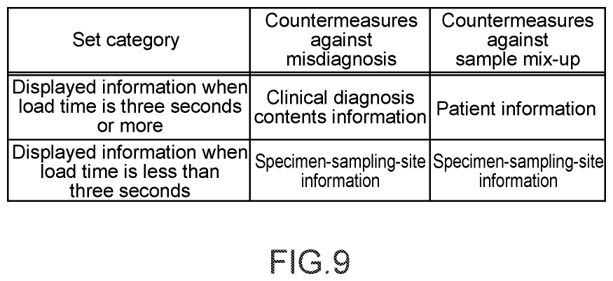

FIG. 8 is a flowchart showing a flow of processing of displaying a WSI image of the DPI system in this case. FIG. 9 is a diagram showing an example of a load time and a user setting table.

For example, the image management server 200 calculates an estimated load time on the basis of the file size of the selected WSI image and the communication speed (bandwidth) of the network interface unit 18, and notifies the viewer computer of the calculated load time (Step 82). The CPU 11 of the viewer computer 100 selects the related information by referring to the calculated load time and a user setting table including the load time and the category of the related information as shown in FIG. 9 (Step 84), and loads and displays it (Step 85). Other processes are the same as those shown in FIG. 4.

In the example of FIG. 9, the clinical diagnosis contents information is set as the related information by the user if the load time is three seconds or more and further if emphasis is placed on the countermeasures against misdiagnoses, and the patient information is set as the related information by the user if emphasis is placed on the countermeasures against sample mix-up. If the load time is less than three seconds, the specimen-sampling-site information is set as the related information regardless of the category. Of course, the method of dividing the load time and setting the category is not limited to this.

(Modification 4 Regarding Method of Displaying Related Information: Timing of Switching from Related Information to WSI Image)

If the check of the related information during the loading of the WSI image is made mandatory as countermeasures in the hospital, it is problematic when a WSI image is displayed before the pathologist checks the related information. In this regard, for example, even if the download of the WSI image has been completed, the viewer computer 100 may be configured not to make a transition of the screen to the WSI until the pathologist presses a button for screen transition, such as “next”, after the related information is displayed. In other words, when the loading of the WSI image is completed and when an operation for displaying that WSI pathological image is received from the user, the viewer computer 100 switches the related information to the WSI image and displays it. As a result, the information processing system can make a transition to the display of the next WSI image after the pathologist checks the related information with certainty by requesting an explicit operation to switch the WSI image.

(Modification Regarding Related Information to be Displayed)

Since a pathologist who is not familiar with the DPI may not know an operation method for the viewer computer 100 (controller 20), the viewer computer 100 may display information regarding an operation method for a WSI image that the pathologist will perform next, as related information, while loading the WSI image. The viewer computer 100 can also learn the operation method of the pathologist (the operation history of the controller 20), and can update the display contents during the loading of the WSI image from the next time.

Specific examples of the information regarding the operation method include a text explaining how to look at a sample (pathology), an explanation for a specific tool such as pressing a zoom button in order to zoom, or enlightening contents such as checking the patient's name and then looking at the image.

At present, the application of the artificial intelligence (AI) technology to the pathology is expected. The AI is a calculation method using, for example, a machine learning model. The machine learning model has parameters generated on the basis of learning data for a specific task, and performs output in accordance with the input data input to the machine learning model. The learning data has a data format similar to that of the input data, for example, and is a plurality of WSI images labeled with pathological results or a plurality of divided pathological images, for example. The machine learning model is, for example, a multi-layer neural network, and is a network model having an input layer, a plurality of intermediate layers, and an output layer. The AI is a machine learning model having parameters generated by learning a plurality of pathological images labeled with diagnosis results such as the presence or absence of a disease and the type of a disease, and it is favorable to output the estimated presence or absence of a disease and type of a disease to an input pathological image. For example, after imaging, the DPI system may store a diagnosis result or the like by the AI in association with the WSI image stored in the image management server 200 before the pathologist refers to the WSI image, and may display it as related information while loading the WSI image.

In this case, when a first WSI image being displayed is switched to a second WSI image and displayed, if the related information is displayed during loading of the second WSI image, the viewer computer 100 may display, as the related information, information indicating the diagnosis result by the AI regarding the second WSI image to be displayed. The information indicating the diagnosis result includes, for example, information regarding the presence or absence of a lesion, the type thereof, the position (annotation, segmentation), the number of cells with a deformity, the name of the diagnosed disease, the display portion until the diagnosis is made, the display order, and the like.

As a result, the viewer computer 100 allows the pathologist to know a corresponding diagnosis result by the AI as a preliminary knowledge before performing a pathological diagnosis regarding the second WSI image during the loading of the second WSI image, so that it is possible for the pathologist to be ready for the place to be viewed in the pathological diagnosis, the display order, or the like and to prevent a misdiagnosis.

In addition, the viewer computer 100 may display information indicating a diagnosis result by the AI regarding the first WSI image that has already been displayed during loading of the second WSI image.

As a result, the viewer computer 100 allows the pathologist to know a corresponding diagnosis result by the AI after the pathological diagnosis regarding the first WSI image, thus preventing a misdiagnosis after the fact. In other words, although there is a possibility that a prejudice occurs when the pathologist views the diagnosis result by the AI before performing a pathological diagnosis, if a corresponding diagnosis result by the AI is displayed after the first WSI image is displayed, the viewer computer 100 can cause the pathologist to check the answer with the diagnosis result without prejudice.

In addition, the information regarding color calibration of the WSI image may be displayed as the related information. In other words, if the color, brightness, contrast, or the like of the WSI image can be set as the related information in accordance with the preference of the pathologist, information indicating the current set value, a sample image, or the like may be displayed as the related information. This allows the pathologist to quickly find a color shift from the color of the standard slide.

Further, as the related information, information in the hospital to be notified to the pathologist (such as information regarding each device in the DPI system) may be displayed.

Further, the related information is not limited to information in the hospital. For example, the information management server 600 may download external information such as diagnosis results of similar cases of prominent physicians of other hospitals to the HD3 in advance, and the viewer computer 100 may load and display those pieces of information as the related information. Further, when a WSI image is selected, the viewer computer 100 may retrieve information on the cloud in real time and display the retrieved information as the related information.

(Modification Regarding Condition for Displaying Related Information)

In the embodiment described above, the viewer computer 100 constantly displays the related information when a load request for a WSI image is made. However, if the load request for the second WSI image instead of the first WSI image is transmitted, the viewer computer 100 may display the related information when the sample or patient serving as an imaging source of the first WSI image is different from the sample or patient serving as an imaging source of the second WSI image, or may not display the related information when the sample or patient is the same.

In this case, if the second WSI image for which a load request is to be transmitted is selected by the pathologist, the viewer computer 100 acquires information regarding the sample or patient serving as the imaging source from the image management server 200 or the information management server 600, and compares such information with the information of the sample or patient of the first WSI image that has been displayed so far.

Therefore, the viewer computer 100 does not constantly display the related information every time the WSI image is switched, but displays the related information only when a precondition of a sample or patient serving as an imaging source is changed, so that it is possible to provide minimum necessary information for the diagnosis while preventing the pathologist from feeling troublesome.

(Modification Regarding Load Source of WSI Image and Related Information)

In the above embodiment, an example in which the viewer computer 100 downloads the WSI images from the image management server 200 in the hospital has been described. However, the load source of the WSI images is not limited to the server. The present technology can be similarly applied to, for example, a case where the WSI is downloaded in advance from the image management server 200 to a storage device (HDD or flash memory) connected to the viewer computer 100 by a cable or the like, and is loaded from that storage device.

[Others]

The present technology can have the following configurations.
(1) An information processing system, including:

a communication unit that transmits a request to load a second pathological image different from a first pathological image being displayed; and

a controller that causes a display unit to display related information related to a pathological diagnosis or an operation of a pathology medical device before the loaded second pathological image is displayed.
(2) The information processing system according to (1), in which

the controller displays the related information during loading of the second pathological image.
(3) The information processing system according to (1) or (2), in which

the controller switches the related information to the second pathological image when loading of the second pathological image is completed.
(4) The information processing system according to (1) or (2), in which

the controller switches the related information to the second pathological image for display when loading of the second pathological image is completed and when an operation for displaying the second pathological image is received from a user.
(5) The information processing system according to any one of (1) to (4), in which

the controller acquires load time information indicating a time necessary for loading the second pathological image and displays the related information of a different type or amount in accordance with the load time information.
(6) The information processing system according to any one of (1) to (5), in which

the controller displays, as the related information, information regarding the second pathological image.
(7) The information processing system according to (6), in which

the controller displays, as the related information, information indicating a clinical diagnosis result corresponding to the second pathological image.
(8) The information processing system according to (6), in which

the controller displays, as the related information, information indicating a sampling site of a specimen serving as an imaging source of the second pathological image.
(9) The information processing system according to (6), in which

the controller displays, as the related information, information indicating a patient serving as an imaging source of the second pathological image.
(10) The information processing system according to any one of (1) to (9), in which

the controller displays, as the related information, information indicating a diagnosis result by artificial intelligence (AI) regarding the first pathological image.
(11) The information processing system according to any one of (1) to (9), in which

the controller displays, as the related information, information indicating a diagnosis result by artificial intelligence (AI) regarding the second pathological image.
(12) The information processing system according to any one of (1) to (9), in which

the controller acquires first sample information indicating a sample serving as an imaging source of the first pathological image and second sample information indicating a sample serving as an imaging source of the second pathological image, and displays the related information when the first sample information and the second sample information are different from each other.
(13) The information processing system according to any one of (1) to (9), in which

the controller acquires first patient information indicating a patient serving as an imaging source of the first pathological image and second patient information indicating a patient serving as an imaging source of the second pathological image, and displays the related information when the first patient information and the second patient information are different from each other.
(14) An information processing method, including:

transmitting a request to load a second pathological image different from a first pathological image being displayed; and

causing a display unit to display related information related to a pathological diagnosis or an operation of a pathology medical device before the loaded second pathological image is displayed.
(15) A program causing an information processing apparatus to execute the steps of:

transmitting a request to load a second pathological image different from a first pathological image being displayed; and

causing a display unit to display related information related to a pathological diagnosis or an operation of a pathology medical device before the loaded second pathological image is displayed.

### REFERENCE SIGNS LIST

11 CPU
13 RAM
14 operation input unit
16 display unit
17 storage unit
18 network interface unit
100 viewer computer
200 image management server
300 digital microscope
400 scanner computer
500 scanner
600 information management server

1. An information processing system, comprising:
a communication unit that transmits a request to load a second pathological image different from a first pathological image being displayed; and
a controller that causes a display unit to display related information related to a pathological diagnosis or an operation of a pathology medical device before the loaded second pathological image is displayed.

2. The information processing system according to claim 1, wherein
the controller displays the related information during loading of the second pathological image.

3. The information processing system according to claim 1, wherein
the controller switches the related information to the second pathological image when loading of the second pathological image is completed.

4. The information processing system according to claim 1, wherein
the controller switches the related information to the second pathological image for display when loading of the second pathological image is completed and when an operation for displaying the second pathological image is received from a user.

5. The information processing system according to claim 1, wherein
the controller acquires load time information indicating a time necessary for loading the second pathological image and displays the related information of a different type or amount in accordance with the load time information.

6. The information processing system according to claim 1, wherein
the controller displays, as the related information, information regarding the second pathological image.

7. The information processing system according to claim 6, wherein
the controller displays, as the related information, information indicating a clinical diagnosis result corresponding to the second pathological image.

8. The information processing system according to claim 6, wherein
the controller displays, as the related information, information indicating a sampling site of a specimen serving as an imaging source of the second pathological image.

9. The information processing system according to claim 6, wherein
the controller displays, as the related information, information indicating a patient serving as an imaging source of the second pathological image.

10. The information processing system according to claim 1, wherein
the controller displays, as the related information, information indicating a diagnosis result by artificial intelligence (AI) regarding the first pathological image.

11. The information processing system according to claim 1, wherein
the controller displays, as the related information, information indicating a diagnosis result by artificial intelligence (AI) regarding the second pathological image.

12. The information processing system according to claim 1, wherein
the controller acquires first sample information indicating a sample serving as an imaging source of the first pathological image and second sample information indicating a sample serving as an imaging source of the second pathological image, and displays the related information when the first sample information and the second sample information are different from each other.

13. The information processing system according to claim 1, wherein
the controller acquires first patient information indicating a patient serving as an imaging source of the first pathological image and second patient information indicating a patient serving as an imaging source of the second pathological image, and displays the related information when the first patient information and the second patient information are different from each other.

14. An information processing method, comprising:
transmitting a request to load a second pathological image different from a first pathological image being displayed; and
causing a display unit to display related information related to a pathological diagnosis or an operation of a pathology medical device before the loaded second pathological image is displayed.

15. A program causing an information processing apparatus to execute the steps of:
transmitting a request to load a second pathological image different from a first pathological image being displayed; and
causing a display unit to display related information related to a pathological diagnosis or an operation of a pathology medical device before the loaded second pathological image is displayed.
